(19) United States
(12) Patent Application Publication
(10) Pub. No.: US 2022/0151685 A1
(43) Pub. Date: May 19, 2022

(54) SURGICAL DEVICE AND METHOD USING TUNGSTEN DISULFIDE

(71) Applicants: Kester Julian Batchelor, Mound, MN (US); Teo Heng Jimmy Yang, Heath (GB)

(72) Inventors: Kester Julian Batchelor, Mound, MN (US); Teo Heng Jimmy Yang, Heath (GB)

(21) Appl. No.: 17/525,268

(22) Filed: Nov. 12, 2021

Related U.S. Application Data
(60) Provisional application No. 63/113,455, filed on Nov. 13, 2020.

(51) Int. Cl. A61B 18/14

(52) U.S. Cl. CPC A61B 18/1445; A61B 2018/00148

(57) ABSTRACT

A surgical device and associated methods are disclosed. In one example, the surgical device includes tungsten disulfide covering all or a portion of a component.

### CLAIM OF PRIORITY

This patent application claims the benefit of priority, under 35 U.S.C. § 119(e), to U.S. Provisional Patent Application Ser. No. 63/113,455, entitled “SURGICAL DEVICE AND METHOD USING TUNGSTEN DISULFIDE,” filed on Nov. 13, 2020, which is hereby incorporated by reference herein in its entirety.

### TECHNICAL FIELD

Embodiments described herein generally relate to medical devices. Specific examples of medical devices include, but are not limited to, forceps, debriders, and lithotripters.

### BACKGROUND

Medical devices for diagnosis and treatment, such as forceps, are often used for medical procedures such as laparoscopic and open surgeries. Forceps can be used to manipulate, engage, grasp, or otherwise affect an anatomical feature, such as a vessel or other tissue of a patient during the procedure. Forceps often include an end effector that is manipulatable from a handle of the forceps. For example, jaws located at a distal end of a forceps can be actuated via elements of the handle between open and closed positions to thereby engage the vessel or other tissue. Forceps can include an extendable and retractable blade that can be extended distally between a pair of jaws to lacerate the tissue. The handle can also be capable of supplying an input energy, such as electromagnetic energy or ultrasound, to the end effector for sealing of a vessel or tissue during a procedure. One technical challenge with medical devices such as forceps includes adhesion of tissue, for example adhesion of tissue after coagulation or cauterization. Improved forceps and other medical devices are desired.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 shows an electrosurgical forceps in accordance with some example embodiments.

FIG. 2A shows a jaw portion of an electrosurgical forceps in accordance with some example embodiments.

FIG. 2B shows a jaw portion of another electrosurgical forceps in accordance with some example embodiments.

FIG. 2C shows a jaw portion of another electrosurgical forceps in accordance with some example embodiments.

### DESCRIPTION OF EMBODIMENTS

The following description and the drawings sufficiently illustrate specific embodiments to enable those skilled in the art to practice them. Other embodiments may incorporate structural, logical, electrical, process, and other changes. Portions and features of some embodiments may be included in, or substituted for, those of other embodiments. Embodiments set forth in the claims encompass all available equivalents of those claims.

The following disclosure may be used with a number of different types of surgical devices. Tungsten disulfide is a low friction material with minimal or no chemical interactivity. These properties make tungsten disulfide desirable for coatings on surgical devices. In one example, tungsten disulfide as combined with other coating layers to provide improved adhesion to desired surfaces. An intermediate layer may be used such that the intermediate layer adheres well to both the substrate and the subsequent tungsten disulfide.

In one example, tungsten disulfide is coated over a chromium nitride layer to provide a coating with both corrosion resistance and anti-stick properties. The chromium nitride protects a substrate material from corrosion and abrasion, while the additional tungsten disulfide provides low friction and anti-stick properties. In one example, tungsten disulfide is coated over a chromium aluminum nitride layer. Chromium aluminum nitride may be used to provide a hard wear resistant property, while the subsequent tungsten disulfide provides low friction and anti-stick properties. In one example, tungsten disulfide is coated over a titanium nitride layer to provide a coating with both corrosion resistance and anti-stick properties. In one example, a combination of chromium nitride, titanium nitride, and tungsten disulfide provides unique anti-stick properties with a contribution from each material coating. For example, titanium nitride may show low adhesion to blood, while chromium nitride may show low adhesion to water, and tungsten disulfide may show low friction to mechanical abrasion. Combinations of some or all of these coatings may provide benefits of each coating in a single device surface.

In one example a tailored amount of a tungsten disulfide property is desired. Properties of tungsten disulfide include, but are not limited to reduced friction, anti-stick, low chemical reactivity, etc. In one example tungsten disulfide may be applied in a pattern to provide a selected level a property provided by the tungsten disulfide. For example a checkered pattern of coated and non-coated regions will have a bulk property that is an average of the surface area of coated and uncoated portions of the checkered pattern.

In one example, a gradient of thickness may be applied to further select an amount of the desired tungsten disulfide property. In one example, a thicker tungsten disulfide coating provides more or less of the desired property, and by varying a coating thickness, an amount of the property is selected for the desired surface.

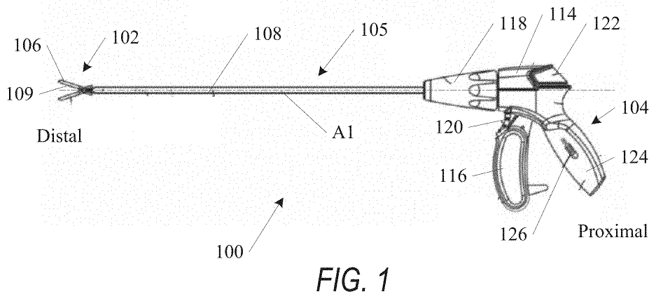

One example surgical device utilizing tungsten disulfide is an electrosurgical forceps as shown in FIG. 1.

FIG. 1 illustrates a side view of a forceps 100 showing jaws in an open position. The forceps 100 can include an end effector 102, a handpiece 104, and an intermediate portion 105. The end effector 102 can include jaws 106 (including electrodes 109), a shaft 108 is shown located between the end effector 102 and the handpiece 104. In one example, the shaft 108 includes, an inner shaft and an outer shaft, and a blade assembly, although the invention is not so limited. The handpiece 104 can include a housing 114, a lever 116, a rotational actuator 118, a trigger 120, an activation button 122, a handle 124, and a locking mechanism 126. FIG. 1 shows orientation indicators Proximal and Distal and a longitudinal axis A1.

Generally, the handpiece 104 can be located at a proximal end of the forceps 100 and the end effector 102 can be located at the distal end of the forceps 100. The intermediate portion 105 can extend between the handpiece 104 and the end effector 102 to operably couple the handpiece 104 to the end effector 102. Various movements of the end effector 102 can be controlled by one or more actuation systems of the handpiece 104. For example, the end effector 102 can be rotated about the longitudinal axis A1 of the forceps 100. Also, the handpiece can operate the jaws 106, such as by moving the jaws 106 between open and closed position. The handpiece 104 can also be used to operate a cutting blade (not shown) for cutting tissue. The handpiece 104 can also be used to operate the electrode 109 for applying electromagnetic energy to tissue. The end effector 102, or a portion of the end effector 102 can be one or more of: opened, closed, rotated, extended, retracted, and electromagnetically energized.

The housing 114 can be a frame that provides structural support between components of the forceps 100. The housing 114 is shown as housing at least a portion of the actuation systems associated with the handpiece 104 for actuating the end effector 102. However, some or all of the actuation components need not necessarily be contained within the housing 114.

A proximal portion of the trigger 120 can be connected to the blade shaft 112b within the housing 114. A distal portion of the trigger 120 can extend outside of the housing 114 adjacent, and in some examples, nested with the lever 116 in the default or unactuated positions. The activation button 122 can be coupled to the housing 114 and can include or be connected to electronic circuitry within the housing 114. Such circuitry can send or transmit electromagnetic energy through the shaft 108 to the electrodes 109. In some examples, the electronic circuitry may reside outside the housing 114 but may be operably coupled to the housing 114 and the end effector 102.

In operation of the forceps 100, a user can displace the lever 116 proximally to drive the jaws 106 from an open position to a closed position, which can allow the user to clamp down on and compress a tissue. The handpiece 104 can also allow a user to move the rotational actuator 118 to cause the end effector 102 to rotate, such as by rotating the shaft 108, or inner components associated with the shaft 108.

In some examples, with the tissue compressed, a user can depress the activation button 122 to cause electromagnetic energy, or in some examples, ultrasound, to be delivered to one or more components of the end effector 102, such as electrodes 109 and in turn to a tissue. Application of such energy can be used to seal or otherwise affect the tissue. In some examples, the electromagnetic energy can cause tissue to be coagulated, sealed, ablated, or can cause controlled necrosis.

In some examples, the handpiece 104 can enable a user to extend and retract a blade (not shown), which can be attached to a distal end of a blade shaft. In some examples, the blade shaft can extend an entirety of a length between the handle 104 and the end effector 102. The blade can be extended by displacing the trigger 120 proximally and the blade can be retracted by allowing the trigger 120 to return distally to a default position.

The forceps 100 can be used to perform a treatment on a patient, such as a surgical procedure. In one example, a distal portion of the forceps 100, including the jaws 106, can be inserted into a body of a patient, such as through an incision or another anatomical feature of the patient's body. While a proximal portion of the forceps 100, including housing 114 remains outside the incision or another anatomical feature of the body. Actuation of the lever 116 causes the jaws 106 to clamp onto a tissue. The rotational actuator 118 can be rotated via a user input to rotate the jaws 106 for maneuvering the jaws 106 at any time during the procedure. Activation button 122 can be actuated to provide electrical energy to jaws 106 to cauterize or seal the tissue within closed jaws 106. Trigger 120 can be moved to translate a blade assembly distally in order to cut tissue within the jaws 106.

In some examples, the forceps 100, or other medical device, may not include all the features described or may include additional features and functions, and the operations may be performed in any order. The handpiece 104 can be used with a variety of other end effectors to perform other methods.

In one example, one or more surfaces of the forceps are coated with tungsten disulfide (WS₂). In one example, one or more portions of tissue contacting surfaces, such as jaws 106, are coated with tungsten disulfide.

In one example, one or more sensors are included at the end effector 102. In one example, where electromagnetic energy is applied to tissue, it is useful to include one or more sensors to indicate when a desired condition of tissue has been achieved. For example, an electrical sensor may be used to measure a property such as resistance of tissue or a region adjacent to tissue. Changes in measured resistance may indicate a state of the environment at the distal end of the end effector, such as coagulation of fluids, level of cauterization of tissue, etc. Other electrical detection data apart from resistance may also be measured by sensors. For example, induction circuits may measure a state of the environment at the distal end of the end effector as a result of a change in a dielectric property of surrounding media. An open circuit detection may indicate a lack of conductivity between two electrodes as a result of a change in state of the environment at the distal end of the end effector.

In one example, maintaining a consistent baseline level for sensors in an environment where electromagnetic energy is applied is challenging. Adhesion of tissues or fluids, or coagulated material, etc. may reduce effectiveness of the sensors. In one example, tungsten disulfide is applied to all or portions of sensors to reduce or eliminate interference with sensor accuracy.

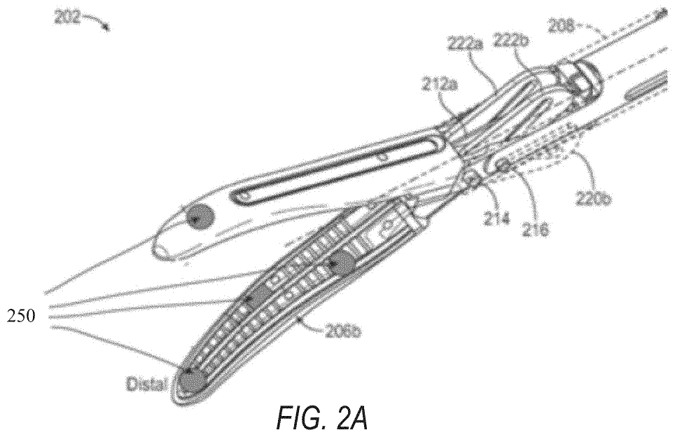

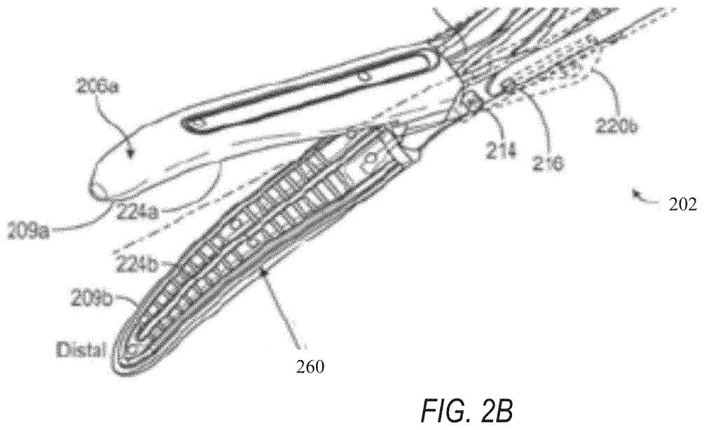

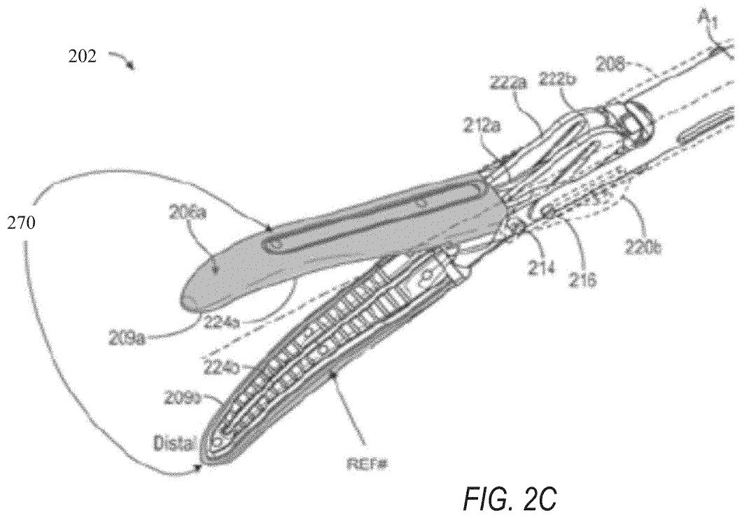

An example forceps, such as forceps 100 from FIG. 1, can include an example end effector 202 as illustrated in FIG. 2A, that can be connected to a handle (such as the handle 104). The end effector 202 can include jaws 206a and 206b, an outer shaft 208, grip plates 209a and 209b, an inner shaft 210, a blade assembly, a pivot pin 214, a drive pin 216, and a guide pin 218. The jaw 206a can include flanges 220a and 220b, and the jaw 206b can include flanges 222a and 222b. The grip plate 209a can include a blade slot 224a and the grip plate 209b can include a blade slot 224b. The blade assembly can include a blade 212a and a shaft. FIGS. 2A-2C also show orientation indicators Proximal and Distal and a longitudinal axis A1.

FIG. 2A-2C show close up views of different examples of jaw configurations. In FIG. 2A, the jaws 206a and 206b (collectively referred to as jaws 206) can be rigid or semi-rigid members configured to engage tissue. The jaws 206a and 206b can be coupled to the outer shaft 208, such as pivotably coupled, via the pivot pin 214. The pivot pin 214 can extend through a portion of the jaws 206a and 206b (such as a bore of each of the jaws 206a and 206b) such that the pivot pin 214 can be received by outer arms of the outer shaft 208. In other examples, the jaws 206a and 206b can be pivotably coupled to the outer shaft 208 via a boss or bosses of the outer shaft 208. In another example, the jaws 206a and 206b can include a boss (or bosses) receivable in bores of the outer shaft 208 to pivotably couple the jaws 206a and 206b to the outer shaft 208. In another example, outer shaft 208 can include a boss (or bosses) receivable in bores of the jaws 206a and 206b to pivotably couple the jaws 206a and 206b to the outer shaft 208.

The flanges 220a and 220b (which can be a set of flanges, that is, two flanges) can be rigid or semi-rigid members located at a proximal portion of the jaw 206a. Similarly, the flanges 222a and 222b can be rigid or semi-rigid members located at a proximal portion of the jaw 206b. In some examples, the flanges 220 can be positioned laterally outward of the inner flanges 222. In other examples, the flanges 220 and 222 can be interlaced.

The grip plates 209a and 209b of the jaws 206a and 206b can each be a rigid or semi-rigid member configured to engage tissue and/or the opposing jaw to grasp tissue, such as during an electrosurgical procedure. One or more of the grip plates 209a and 209b can include one or more of serrations, projections, ridges, or the like configured to increase engagement pressure and friction between the grip plates 209a and 209b and tissue. The flanges 220 of the upper jaw 206a can extend proximally away from the grip plate 209a and 209b, and in some examples, substantially downward when the upper jaw 206a is in the open and partially open positions. Similarly, the flanges 222 of the lower jaw 206b can extend proximally away from the grip plate, and in some examples, substantially upward when the upper jaw 206a is in the open and partially open positions, such that the jaws 206a and 206b and flanges 220 and 222 operate to open and close in a scissoring manner.

The jaws 206a and 206b can each include an electrode configured to deliver electricity to tissue (optionally through the grip plates 209a and 209b), and a frame supporting the electrode. The blade slots 224a and 224b of the grip plates 209a and 209b can together be configured to receive a blade between the jaws 206a and 206b, when the jaws are moved out of the open position. In some examples, only one blade slot may be used.

Each of the inner shaft 210 and the outer shaft 208 can be a rigid or semi-rigid and elongate body having a geometric shape of a cylinder, where the shape of the inner shaft 210 matches the shape of the outer shaft 208. In some examples, the inner shaft 210 and the outer shaft 208 can have other shapes such as an oval prism, a rectangular prism, a hexagonal prism, an octagonal prism, or the like. In some examples, the shape of the inner shaft 210 can be different from the shape of the outer shaft 208.

The inner shaft 210 can extend substantially proximally to distally along the axis A1, which can be a longitudinal axis. In some examples, the axis A1 can be a central axis. Similarly, the outer shaft 208 can extend substantially proximally to distally along the axis A1. In some examples, the axis A1 can be a central axis of one or more of the inner shaft 210 and the outer shaft 208. The inner shaft 210 can include an axial bore extending along the axis A1. The outer shaft 208 can also include an axial bore extending along the axis A1. The inner shaft 210 can have an outer dimension (such as an outer diameter) smaller than an inner diameter of the outer shaft 208 such that the inner shaft 210 can be positioned within the outer shaft 208 and such that the inner shaft 210 can be translatable in the outer shaft 208 along the axis A1. The inner shaft 210 can also be referred to as a drive shaft 210, a cam shaft 210, or an inner tube 210. The outer shaft 208 can also be referred to as an outer tube 208.

The blade 212a can be an elongate cutting member at a distal portion of the blade assembly. The blade 212a can include one or more sharpened edges configured to cut or resect tissue or other items. The blade assembly 12 can be located within the outer shaft 208 (and can be located within the inner shaft 210). The blade 212a can extend along (and optionally parallel with) the axis A1. The blade 212a can be translatable with respect to the inner shaft 210 and the outer shaft 208 to extend between (or into) the first jaw 206a and the second jaw 206b, such as along the blade slots 224a and 224b. In some examples, the blade 212a can extend axially through the inner shaft 210 offset from the axis A1. In some examples, the blade 212a can extend axially through the flanges 220 and 222 such that the blade 212a is in a position laterally inward of the first set of flanges 220 and the second set of flanges 222. The blade 212a can also be a translating member or electrosurgical component other than a blade. For example, the translating member 212a can be an electrode, such as a blunt electrode, a needle electrode, or a snare electrode.

The guide 218, the drive pin 216, and the pivot pin 214 can each be a rigid or semi-rigid pin, such as a cylindrical pin. The guide 218, the drive pin 216, and the pivot pin 214 can have other shapes in other examples, such as rectangular, square, oval, or the like. In some examples, the pins can all be of the same size but can be different sizes in other examples. Each pin can have a smooth surface to help reduce surface friction between the pins and components of the forceps 200, such as between the pivot pin 214 and the outer shaft 208 or the drive pin 216 and the flanges 220 and 222. Each of the guide 218, the drive pin 216, and the pivot pin 214 can be other components such as one or more projections, bosses, arms, or the like.

In operation, the inner shaft 210 can be translated using an actuator (such as the lever 116 of FIG. 1). The inner shaft 210 can translate with respect to the outer shaft 208 to move the drive pin 216. The drive pin 216 can engage the flanges 220 and 222 to move the flanges 220 and 222 between open and closed positions, which can cause the jaws 206a and 206b to pivot about the pivot pin 214 (such as with respect to the inner shaft 210, the outer shaft 208, or the blade 212) to move the jaws 206 between open and closed positions.

As described above, in one example, the addition of one or more sensors on the end effector 202 is useful to determine a state of the environment at the distal end of the end effector. FIG. 2A shows one example of locations for sensors 250. In one example one or more of sensors 250 include at least a partial coating of tungsten disulfide. In the example of FIG. 2A, the sensors 250 are distinct separate components from jaw surfaces.

FIG. 2B shows one example of locations for sensors 260. In one example one or more of sensors 260 include at least a partial coating of tungsten disulfide. In contrast to the individual isolated sensor elements 250 of FIG. 2A, the one or more sensors 260 of FIG. 2B may be incorporated into a component of the jaws, such as a contact surface. In one example, one or more jaw surfaces serve a dual function of providing electromagnetic energy and sensing.

FIG. 2C shows one example of locations for sensors 270. In one example one or more of sensors 270 include at least a partial coating of tungsten disulfide. In the example of FIG. 2B, the sensor 270 shown is broader, and may include a large surface or an entire surface of one or more jaws. In one example, a housing portion of one or more jaws provides a sensing function, while contact surfaces between the jaws provide application of electromagnetic energy to heat tissue.

In one example, tungsten disulfide may be doped with conductive metal particles to provide a desired resistance or conductivity to the tungsten disulfide coating. In one example, tungsten disulfide may be doped with silver particles. In combination with sensors as described above, it can be advantageous to have both conductivity and anti-stick properties when used in conjunction with an electrical sensor.

Forceps are shown as one example of a surgical device where a tungsten disulfide coating provides low friction and anti-stick advantages. One of ordinary skill in the art, having the benefit of the present disclosure, will recognize that other surgical devices will also benefit from the addition of tungsten disulfide coatings and composite coatings. For example, other surgical devices that utilize sensors will benefit from coatings of tungsten disulfide. Other devices that utilize heat and may encounter tissue sticking issues will benefit. For example, selected components of laser instruments will benefit from coatings including tungsten disulfide.

To better illustrate the method and apparatuses disclosed herein, a non-limiting list of embodiments is provided here:

Example 1 includes a forceps. The forceps includes jaws located at an end of a shaft, a jaw actuator routed along the shaft and coupled to one or more of the jaws, a pair of electrodes coupled to opposing surfaces of jaws, one or more sensors located on the jaws, and a tungsten disulfide coating on at least a portion of the one or more sensors.

Example 2 includes a surgical device. The surgical device includes two surfaces that are configured to move with respect to one another, and a tungsten disulfide coating on at least a portion of one or more of the two surfaces, wherein the tungsten disulfide coating is non-uniform across an interface between the two surfaces.

Example 3 includes a surgical device. The surgical device includes a coated surface on at least a portion of a component of the surgical device, wherein the coated surface includes a tungsten disulfide layer, an intermediate coating between the tungsten disulfide layer and the portion of the component.

Example 4 includes a surgical device. The surgical device includes tungsten disulfide at least partially covering a component of the surgical device, wherein the tungsten disulfide is configured according to an example of the present disclosure.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Although an overview of the inventive subject matter has been described with reference to specific example embodiments, various modifications and changes may be made to these embodiments without departing from the broader scope of embodiments of the present disclosure. Such embodiments of the inventive subject matter may be referred to herein, individually or collectively, by the term “invention” merely for convenience and without intending to voluntarily limit the scope of this application to any single disclosure or inventive concept if more than one is, in fact, disclosed.

The embodiments illustrated herein are described in sufficient detail to enable those skilled in the art to practice the teachings disclosed. Other embodiments may be used and derived therefrom, such that structural and logical substitutions and changes may be made without departing from the scope of this disclosure. The Detailed Description, therefore, is not to be taken in a limiting sense, and the scope of various embodiments is defined only by the appended claims, along with the full range of equivalents to which such claims are entitled.

As used herein, the term “or” may be construed in either an inclusive or exclusive sense. Moreover, plural instances may be provided for resources, operations, or structures described herein as a single instance. Additionally, boundaries between various resources, operations, modules, engines, and data stores are somewhat arbitrary, and particular operations are illustrated in a context of specific illustrative configurations. Other allocations of functionality are envisioned and may fall within a scope of various embodiments of the present disclosure. In general, structures and functionality presented as separate resources in the example configurations may be implemented as a combined structure or resource. Similarly, structures and functionality presented as a single resource may be implemented as separate resources. These and other variations, modifications, additions, and improvements fall within a scope of embodiments of the present disclosure as represented by the appended claims. The specification and drawings are, accordingly, to be regarded in an illustrative rather than a restrictive sense.

The foregoing description, for the purpose of explanation, has been described with reference to specific example embodiments. However, the illustrative discussions above are not intended to be exhaustive or to limit the possible example embodiments to the precise forms disclosed. Many modifications and variations are possible in view of the above teachings. The example embodiments were chosen and described in order to best explain the principles involved and their practical applications, to thereby enable others skilled in the art to best utilize the various example embodiments with various modifications as are suited to the particular use contemplated.

It will also be understood that, although the terms “first,” “second,” and so forth may be used herein to describe various elements, these elements should not be limited by these terms. These terms are only used to distinguish one element from another. For example, a first contact could be termed a second contact, and, similarly, a second contact could be termed a first contact, without departing from the scope of the present example embodiments. The first contact and the second contact are both contacts, but they are not the same contact.

The terminology used in the description of the example embodiments herein is for the purpose of describing particular example embodiments only and is not intended to be limiting. As used in the description of the example embodiments and the appended examples, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

1. An electrosurgical forceps, comprising:
a handpiece;
an end effector coupled distally from the handpiece, wherein the end effector includes;
one or more jaws; and
tungsten disulfide at least partially covering a portion of the one or more jaws.

2. The electrosurgical forceps of claim 1, wherein the tungsten disulfide at least partially covers an electrode of the one or more jaws.

3. The electrosurgical forceps of claim 1, wherein the tungsten disulfide at least partially covers an electrical sensor of the one or more jaws.

4. The electrosurgical forceps of claim 3, wherein the electrical sensor is included in a housing portion of the one or more jaws.

5. The electrosurgical forceps of claim 3, wherein the electrical sensor is includes a resistance sensor.

6. The electrosurgical forceps of claim 1, wherein the tungsten disulfide at least partially covers multiple electrical sensors of the one or more jaws.

7. The electrosurgical forceps of claim 1, wherein the tungsten disulfide includes doped tungsten disulfide.

8. The electrosurgical forceps of claim 7, wherein the doped tungsten disulfide includes an electrical property modifying dopant.

9. The electrosurgical forceps of claim 8, wherein the electrical property modifying dopant includes silver.

10. A surgical device, comprising:
two surfaces that are configured to move with respect to one another, and
a tungsten disulfide coating on at least a portion of one or more of the two surfaces;
wherein the tungsten disulfide coating is non-uniform across an interface between the two surfaces.

11. The surgical device of claim 10, wherein the two surfaces include forceps jaws.

12. The surgical device of claim 10, wherein the tungsten disulfide includes doped tungsten disulfide.

13. The surgical device of claim 10, wherein the tungsten disulfide includes an electrical property modifying dopant.

14. The surgical device of claim 10, wherein the electrical property modifying dopant includes silver.

15. An electrosurgical forceps, comprising:
a handpiece;
an end effector coupled distally from the handpiece, wherein the end effector includes;
one or more jaws;
a translating component that is movable with respect to the one or more jaws; and
tungsten disulfide at least partially covering the translating component.

16. The electrosurgical forceps of claim 15, wherein the translating component includes a blade.

17. The electrosurgical forceps of claim 15, wherein the translating component includes an electrode.
